(19) United States
(12) Patent Application Publication
(10) Pub. No.: US 2008/0228169 A1
(43) Pub. Date: Sep. 18, 2008

(54) OVER-THE-WIRE CATHETER WITH LATERAL ACCESS

(76) Inventor: Richard A. Schatz, San Diego, CA (US)

Correspondence Address: NYDEGGER & ASSOCIATES, 348 OLIVE STREET, SAN DIEGO, CA, 92103, US

(21) Appl. No.: 11/685,304

(22) Filed: Mar. 13, 2007

(51) Int. Cl. A61M 25/09

(52) U.S. Cl. 604/528

(57) ABSTRACT

An over-the-wire catheter includes a guide wire lumen that is defined by a distal bracket and a proximal bracket. Both brackets are formed as part of the catheter, and the lumen is dimensioned for receiving a guide wire therethrough. Between the distal bracket and the proximal bracket is a lateral aperture that extends along the length of the catheter and provides for selective access to the guide wire during the use and operation of the catheter.

### FIELD OF THE INVENTION

The present invention pertains generally to catheters that are advanced into the vasculature of a patient to perform a medical/surgical procedure. More particularly, the present invention pertains to systems and methods for guiding a catheter through the vasculature of a patient over a guide wire. The present invention is particularly, but not exclusively, useful as an over-the-wire catheter that is modified to provide an open access for manipulation of its guide wire at a location proximate the distal end of the catheter.

### BACKGROUND OF THE INVENTION

Interventional surgical procedures typically require that a catheter be somehow advanced through the vasculature of a patient to a predetermined site. Catheters, however, are typically designed for a particular purpose that is to be accomplished after the catheter has been positioned in the vasculature. Therefore, as a practical matter, a particular catheter may not necessarily have the structural wherewithal to effectively navigate the vasculature on its own. Consequently, the advancement of a catheter into the vasculature is often accomplished with assistance from other means, such as a pre-positioned guide wire. As can be easily appreciated, the pre-positioning of the guide wire, and the subsequent advancement of the catheter along the guide wire, must be done with the utmost care and precision.

Presently there are two well-known methods for advancing a catheter along a guide wire. These methods are generally known as: 1) the over-the-wire method; and 2) the monorail method. For the first, i.e. the over-the-wire method, the catheter is formed with a guide wire lumen that extends the entire length of the catheter. Thus, the entire catheter follows the wire during its advancement into the vasculature. A significant advantage for this method is that the “pushability” of the catheter is substantially enhanced. On the other hand, for the monorail method, only a portion of the catheter is formed with a guide wire lumen. Accordingly, part of the catheter can remain free of the catheter to enhance the individual manipulability of the catheter.

Depending on the particular medical/surgical procedure that is being performed, both methods of guide wire use have their advantages. It can happen for some specific procedures, however, that the ability to select between an over-the-wire method and a monorail method may be advantageous. For instance, procedures such as a device exchange or the crossing of a bifurcation lesion could easily give rise to the need for such a selection. Moreover, for some procedures it may be desirable to actually change from one method of guide wire use to the other during the procedure.

In light of the above it is an object of the present invention to provide a catheter system that can be selectively used either as an over-the-wire type catheter or as a monorail type catheter. Another object of the present invention is to provide a catheter system that allows the operator (i.e. physician) direct access both to the catheter and to the guide wire at selected locations along the length of the catheter. Still another object of the present invention is to provide a catheter system that is relatively simple to manufacture, is easy to use and is cost effective.

### SUMMARY OF THE INVENTION

A catheter system in accordance with the present invention includes a catheter that is specifically designed to facilitate the advancement of the catheter over a guide wire and into the vasculature of a patient. To do this, the present invention provides a catheter that permits changes in the structural interaction of the catheter with the guide wire. Specifically, the catheter of the present invention allows the operator to selectively change between an over-the-wire and a monorail method of guide wire usage. More generally, the catheter system of the present invention permits an over-the-wire type operation, a monorail type operation, or a hybrid combination of the two. It also allows the operator access to the guide wire, at selected locations along the length of the catheter.

Structurally, the catheter of the present invention has a proximal end and a distal end with a main lumen that extends along the length of the catheter between the two ends. Also, the catheter has a distal bracket and a proximal bracket. Specifically, the distal bracket is formed on the catheter to extend from the distal end of the catheter in a proximal direction. It surrounds and defines a distal guide wire lumen. Similarly, the proximal bracket is formed on the catheter to extend from the proximal end of the catheter in a distal direction. It surrounds and defines a proximal guide wire lumen. A consequence of this is that a lateral access is established between the distal bracket and the proximal bracket. In order to help stabilize a guide wire in the lateral access, the surface of the catheter between the distal bracket and the proximal bracket can be formed with a groove that is dimensioned to receive the guide wire therein.

As envisioned for the present invention, an inflatable angioplasty balloon can be mounted on the distal end of the catheter in fluid communication with the main lumen. Also, a Y-site can be attached to the proximal end of the catheter. If used, one branch of the Y-site is placed in fluid communication with the main lumen. Thus, the Y-site establishes a main port that can be used for an inflation/deflation of the balloon. The other branch of the Y-site can then be used for the guide wire. Specifically, this other branch of the Y-site establishes a proximal end port that connects with the proximal guide wire lumen of the proximal bracket. At the distal end of the catheter, the distal bracket provides a distal end port that allows the guide wire to be introduced into the guide wire lumen of the distal bracket. As indicated above, the proximal and distal guide wire lumens can act in concert with each other to establish a comprehensive guide wire lumen for the catheter.

In an alternate embodiment of the present invention, the catheter can be formed with a central bracket that is located between the distal bracket and the proximal bracket. For this embodiment, the central bracket will effectively divide the lateral access into a proximal lateral access and a distal lateral access. Structurally and functionally, the central bracket is essentially the same as either the proximal bracket or the distal bracket.

In its operation, the present invention envisions that a guide wire will be pre-positioned in the vasculature of a patient. The extracorporeal end of the guide wire can then be inserted into the distal end port of the catheter and received into the distal guide wire lumen. Additional advancement of the catheter over the guide wire will then bring the exposed extracorporeal end of the guide wire past the lateral access and into the vicinity of the proximal bracket. At this point, the guide wire can be selectively inserted into the proximal guide wire lumen of the proximal bracket. If so, the guide wire will exit the proximal bracket through the proximal end port and the catheter can thereafter be used as an over-the-wire catheter. Otherwise, when the guide wire is not passed through the proximal bracket, the guide wire is restrained by only the distal bracket and can stay free from the catheter at the lateral access. In this case, the catheter can be used in a monorail methodology. It will be appreciated that the catheter's method of guide wire usage can be changed at any time during a procedure, either by selectively inserting the guide wire through the proximal bracket (over-the-wire), or not inserting the guide wire through the proximal bracket (monorail).

### BRIEF DESCRIPTION OF THE DRAWINGS

The novel features of this invention, as well as the invention itself, both as to its structure and its operation, will be best understood from the accompanying drawings, taken in conjunction with the accompanying description, in which similar reference characters refer to similar parts, and in which:

FIG. 1 is a perspective view of a catheter system in its intended operational environment;

FIG. 2A is a perspective view of a preferred embodiment of a catheter of the present invention;

FIG. 2B is a view of the catheter shown in FIG. 2A when engaged with a guide wire for an over-the-wire operation;

FIG. 2C is a view of the catheter shown in FIG. 2A when engaged with a guide wire for a monorail operation;

FIG. 3 is a cross section view of the catheter system of the present invention as seen along the line 3-3 in FIG. 2B;

FIG. 4A is a cross section view of the catheter system of the present invention as seen along the line 4-4 in FIG. 2B;

FIG. 4B is a cross section view of the catheter system of the present invention as seen along the line 4-4 in FIG. 2C; and

FIG. 5 is a perspective view of an alternate embodiment of a catheter of the present invention.

### DESCRIPTION OF THE PREFERRED EMBODIMENTS

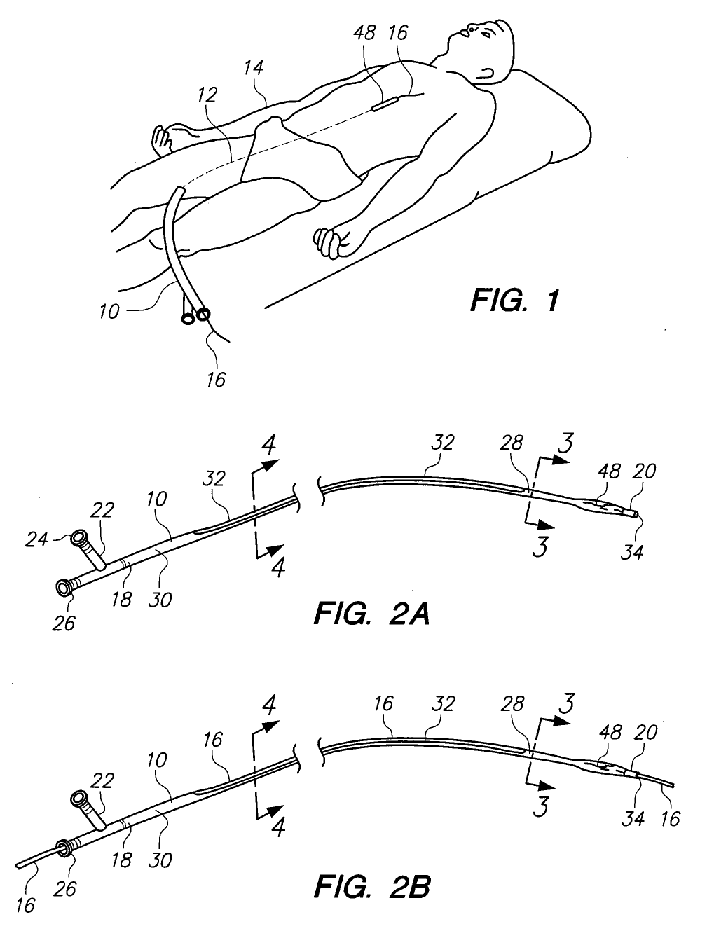

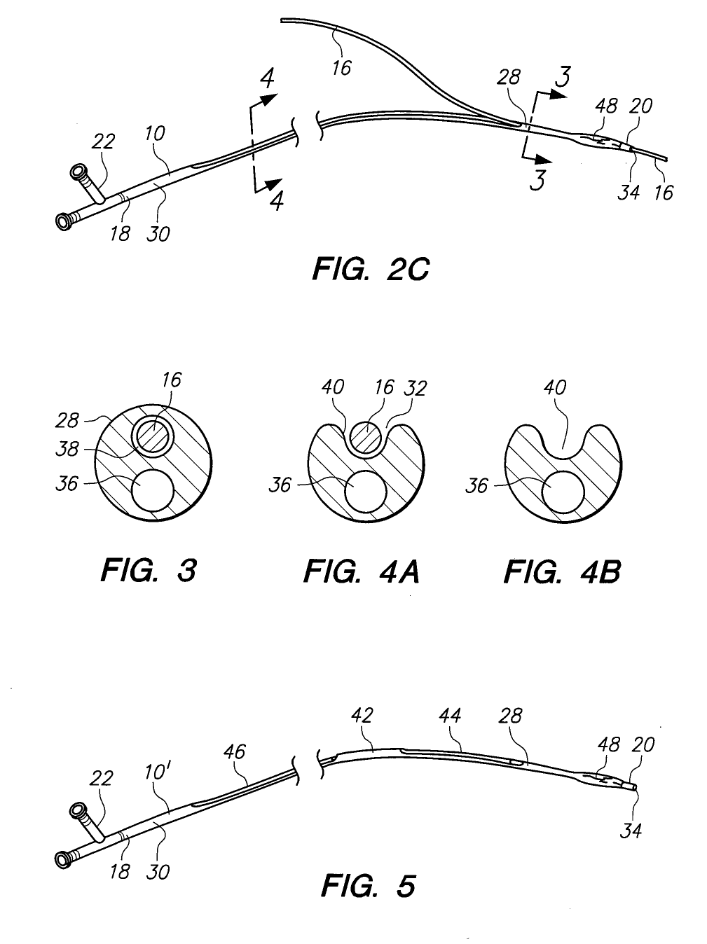

Referring initially to FIG. 1 a catheter in accordance with the present invention is shown and designated 10. As shown, the catheter 10 has been advanced along a path 12 through the vasculature of a patient 14 over a guide wire 16. The actual structure for the catheter 10 will be best seen in FIG. 2A.

In FIG. 2A it will be seen that the catheter 10 has a proximal end 18 and a distal end 20. A Y-site 22, of a type well known in the pertinent art, is attached to the proximal end 18 of the catheter 10 to establish a main port 24 and a proximal end port 26. FIG. 2A also shows that a distal bracket 28 is formed on the catheter 10, and that the distal bracket 28 extends in a proximal direction from the distal end 20 of the catheter 10. Similarly, FIG. 2A shows a proximal bracket 30 that is formed on the catheter 10, and that the proximal bracket 30 extends in a distal direction from the proximal end 18 of the catheter 10. It is also seen in FIG. 2A that, together, the distal bracket 28 and the proximal bracket 30 establish a lateral aperture 32 that extends along the length of the catheter 10, between the brackets 28 and 30.

FIG. 2B shows the catheter 10 engaged with the guide wire 16 for an over-the-wire method of operation. Specifically, in this over-the-wire configuration, it will be seen that the guide wire 16 extends along the entire length of the catheter 10, between the proximal end port 26 and a distal end port 34, and beyond the ports 26 and 34. Further, in the over-the-wire configuration, the guide wire 16 remains next to the catheter 10. The guide wire 16, however, is still accessible in the region of the lateral aperture 32. For a different methodology, as shown in FIG. 2C, the guide wire 16 is restrained by only the distal bracket 28. In this monorail configuration (FIG. 2C), the guide wire 16 still interacts with the catheter 10, and it can still be effectively used to advance the catheter 10, but it can also be manipulated separately from the catheter 10.

FIG. 3 shows that the distal bracket 28 is formed with both a main lumen 36 and a guide wire lumen 38. As will be appreciated by the skilled artisan, the proximal bracket 30 has a substantially same structure as the distal bracket 28. Specifically, the main lumen 36, which extends the entire length of catheter 10, is also present in the proximal bracket 30. Additionally, the proximal bracket 30 is formed with a portion of the guide wire lumen 38.

In detail, for the over-the-wire configuration of catheter 10 (see FIG. 2B) the guide wire 16 passes through the guide wire lumen 38 of the distal bracket 28 (see FIG. 3). Likewise, the guide wire 16 passes through the guide wire lumen 38 that is formed into the proximal bracket 30. Between the two brackets 28 and 30 the guide wire 16 is juxtaposed with the catheter 10 for the over-the-wire configuration. This places it in the lateral aperture 32 as shown in FIG. 2B and FIG. 4A. As will be best appreciated with reference to FIG. 4A, in order to help stabilize the guide wire 16 in the over-the-wire configuration, the catheter 10 can be formed with a groove 40. Specifically, if used, this groove 40 will extend along the side of the catheter 10 between the distal bracket 28 and the proximal bracket 30.

In the monorail configuration for catheter 10 (see FIG. 2C), the guide wire 16 is held on the catheter 10 by only the distal bracket 28. For this configuration, in the region of the lateral aperture 32, the guide wire 16 can be separated from the catheter 10 for individual manipulation (see FIG. 4B). Accordingly, in the monorail configuration, the guide wire 16 does not pass through the proximal bracket 30.

An alternate embodiment for the present invention is shown as the catheter 10′ in FIG. 5. For this embodiment, the catheter 10′ includes a central bracket 42. In all of its important respects, the central bracket 42 is similar to the distal bracket 28 and the proximal bracket 30 disclosed above. Specifically, the central bracket 42 is also formed with a guide wire lumen 38 and a main lumen 36. The central bracket 42 does, however, divide what would otherwise be the lateral aperture 32 into a distal lateral aperture 44 and a proximal lateral aperture 46.

For purposes of this disclosure, the catheter 10 is shown to be a so-called “balloon catheter.” Specifically, in FIG. 2A it is shown that an inflatable balloon 48 is attached to the distal end 20 of the catheter 10. More specifically, the balloon 48 is attached in fluid communication with the main lumen 36. Thus, an inflation/deflation device (not shown) can be attached to the main port 24 of the Y-site 22, and used to inflate/deflate the balloon 48. Such an operation is only exemplary and, as envisioned for the present invention, the catheter 10 can be any of several types of catheters well known in the pertinent art for performing medical/surgical procedures in the vasculature of a patient 14. Importantly, any such procedure is separate and distinct from the functional interaction of the catheter 10 with a guide wire 16.

As envisioned for the present invention, the interaction of the catheter 10 with a guide wire 16 will most often happen after the guide wire 16 has been pre-positioned in the vasculature of the patient 14. To begin, the extracorporeal end of guide wire 16 will first be inserted through the distal end port 34 of the distal bracket 28. As the guide wire 16 then exits from the distal bracket 28, the operator has a choice. One choice is to do no more, and use the catheter 10 in a monorail configuration. The other choice is to insert the guide wire 16 into the guide wire lumen 38 of the proximal bracket 30 until it extends from the proximal end port 26 at the Y-site 22. The operator can then use the catheter 10 in its over-the-wire configuration. As will be appreciated by the skilled artisan, the configurations are selectively interchangeable at any time.

While the particular Over-The-Wire Catheter With Lateral Access as herein shown and disclosed in detail is fully capable of obtaining the objects and providing the advantages herein before stated, it is to be understood that it is merely illustrative of the presently preferred embodiments of the invention and that no limitations are intended to the details of construction or design herein shown other than as described in the appended claims.

What is claimed is:

1. A system for performing an interventional medical procedure in the vasculature of a patient which comprises:
a guide wire pre-positioned in the vasculature; and
a catheter having a proximal end and a distal end, wherein the catheter is formed with a guide wire lumen extending between the proximal end and the distal end, and further wherein the catheter is formed with a lateral aperture extending partway along the guide wire lumen for access to the guide wire in the guide wire lumen, and manipulation thereof, as the catheter is advanced over the guide wire and into the vasculature during the procedure.

2. A system as recited in claim 1 further comprising:
a distal bracket formed on the catheter to surround and define a distal portion of the guide wire lumen, with the distal bracket extending in a proximal direction from the distal end of the catheter; and
a proximal bracket formed on the catheter to surround and define a proximal portion of the guide wire lumen, with the proximal bracket extending in a distal direction from the proximal end of the catheter, to locate the lateral aperture between the distal bracket and the proximal bracket.

3. A system as recited in claim 2 further comprising a central bracket formed on the catheter to surround and define a central portion of the guide wire lumen, wherein the central bracket is positioned between the distal bracket and the proximal bracket for creation of a distal lateral aperture and a proximal lateral aperture.

4. A system as recited in claim 1 wherein the catheter is formed with a main lumen, with the main lumen being substantially parallel to the guide wire lumen.

5. A system as recited in claim 4 wherein the distal end of the catheter is formed with a distal end port for receiving the guide wire into the guide wire lumen.

6. A system as recited in claim 4 wherein the proximal end of the catheter is formed with a proximal end port for allowing the guide wire to extend in a proximal direction from the catheter.

7. A system as recited in claim 4 wherein the proximal end of the catheter is formed with a main port to establish the main lumen as a passageway.

8. A system as recited in claim 1 wherein the catheter has a length “l” and the lateral aperture spans a distance “d”, and wherein “d” is greater than half of “l” (d>0.5l).

9. A system for performing an interventional medical procedure in the vasculature of a patient which comprises:
a guide wire pre-positioned in the vasculature;
a catheter having a proximal end and a distal end;
a distal bracket formed on the catheter to surround and define a distal guide wire lumen, with the distal bracket extending in a proximal direction from the distal end of the catheter; and
a proximal bracket formed on the catheter to surround and define a proximal guide wire lumen, with the proximal bracket extending in a distal direction from the proximal end of the catheter toward the center point of the catheter to establish an open lateral aperture between the distal bracket and the proximal bracket for access to the guide wire, and manipulation thereof, as the catheter is advanced into the vasculature over the guide wire during the procedure.

10. A system as recited in claim 9 wherein the catheter has a length “l” and the aperture spans a distance “d”, and wherein “d” is greater than half of “l” (d>0.5l).

11. A system as recited in claim 9 further comprising a central bracket formed on the catheter to surround and define a central guide wire lumen, wherein the central bracket is positioned between the proximal bracket and the distal bracket for creation of a distal lateral aperture and a proximal lateral aperture.

12. A system as recited in claim 11 wherein the catheter has a length “l” and the distal aperture spans a distance “d_{d}” and the proximal aperture spans a distance “dₛ”, and further wherein d_{d}+dₛ>0.5l.

13. A system as recited in claim 9 wherein the catheter is formed with a main lumen.

14. A system as recited in claim 13 wherein the distal end of the catheter is formed with a distal end port for receiving the guide wire into the guide wire lumen, wherein the proximal end of the catheter is formed with a proximal end port for allowing the guide wire to extend in a proximal direction from the catheter, and wherein the proximal end of the catheter is formed with a main port to establish the main lumen as a passageway.

15. A method for manufacturing a system for performing an interventional medical procedure in the vasculature of a patient which comprises the steps of:
providing a catheter having a proximal end and a distal end with a main lumen extending therebetween;
forming a distal bracket on the catheter to surround and define a distal portion of a guide wire lumen, with the distal bracket extending in a proximal direction from the distal end of the catheter;
forming a proximal bracket on the catheter to surround and define a proximal portion of the guide wire lumen, with the proximal bracket extending in a distal direction from the proximal end of the catheter, to locate a lateral aperture between the distal bracket and the proximal bracket; and
dimensioning the distal portion and the proximal portion of the guide wire lumen for receiving a guide wire therethrough.

16. A method as recited in claim 15 further comprising the step of forming a central bracket on the catheter to surround and define a central portion of the guide wire lumen, wherein the central bracket is positioned between the distal bracket and the proximal bracket for creation of a distal lateral aperture and a proximal lateral aperture.

17. A method as recited in claim 15 further comprising the step of creating a groove along the catheter in the lateral aperture for stabilizing the guide wire between the distal bracket and the proximal bracket.

18. A method as recited in claim 15 further comprising the step of attaching a Y-site to the proximal end of the catheter to establish a proximal end port for the guide wire lumen and to establish a main port for the main lumen.

19. A method as recited in claim 15 further comprising the step of mounting an inflatable balloon at the distal end of the catheter, wherein the balloon is established in fluid communication with the main lumen of the catheter.

20. A method as recited in claim 15 wherein the guide wire is made of stainless steel and the catheter is made of a polymer.
